(19) United States
(12) Patent Application Publication
(10) Pub. No.: US 2007/0014723 A1
(43) Pub. Date: Jan. 18, 2007

(54) Methods and compositions for 10Beryllium complex probes

(76) Inventors: Richard T. Sawyer, Rockville, MD (US); Brian J. Day, Englewood, CO (US); Lee S. Newman, Denver, CO (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 11/486,682

(22) Filed: Jul. 14, 2006

Related U.S. Application Data
(60) Provisional application No. 60/699,085, filed on Jul. 14, 2005.

(51) Int. Cl. A61K 51/00

(52) U.S. Cl. 424/1.11

(57) ABSTRACT

The present invention concerns methods and compositions for making and using Be complexes of defined compositions, which may have multiple functionalities and/or binding specificities. In various embodiment, Beryllium (Be) complexes may include Be such as ¹⁰Be and ⁷Be complexes. Such complexes find use in a wide variety of applications, particularly in the field of treatment, detection and/or diagnosis of infections, diseases and other health-related conditions, including but not limited to cancer, autoimmune disease, cardiovascular disease, metabolic diseases, degenerative diseases, and organ transplant rejection. In addition, a Be complex may be used in a BeLPT assay

### CROSS-REFERENCE TO RELATED APPLICATIONS

The present application claims the benefit under 35 U.S.C. §119(e) of provisional U.S. patent application Ser. No. 60/699,085, filed on Jul. 14, 2005.

### FEDERALLY FUNDED RESEARCH

This invention was made with Government support under Grant Numbers RO1 ES-06538, PO1 ES11810, K08 HL03887 and MO1 RR0051 from the National Institutes of Health. The U.S. government may have certain rights to practice the subject invention.

### FIELD

Various embodiments of the present invention concern methods and compositions for making and using Beryllium (Be) complexes such as ¹⁰Beryllium (¹⁰Be) and ⁷Be complexes. Such complexes may find use in a wide variety of applications, particularly in the field of treatment, detection and/or diagnosis of infections, diseases and other health-related conditions, including, but not limited to cancer, autoimmune disease, cardiovascular disease, metabolic diseases, degenerative diseases, and organ transplant rejection.

### BACKGROUND

Environmental Toxins

Early detection of a disease or a condition is an important aspect in treatment, attenuation and prevention of a disease. In one particular example, exposure to environmental toxins and other macromolecules is critical in the intervention of disease caused by these toxins. Examples of environmental toxins include: 1) Macromolecules derived from a variety of sources including microbial, botanical and man-made; 2) Small inorganic and organic molecules that occur naturally, or which are also man-made, 3) The products of genetic engineering, and 4) Viruses/Prions—including those that are currently known, or those that may be discovered in the future. These environmental toxic substances play a significant role in the pathogenesis of a variety of human disease processes, from cancer, to occupationally and environmentally acquired disease. Examples of viruses/prions include Corona virus (CoV), an agent of Severe Acute Respiratory Syndrome (SARS), a type of environmental toxin induced disease.

Moreover, it has only recently been appreciated that very small amounts of these substances may play a critical role in establishing these disease processes. This realization has been due, in part, to the development of more sensitive analytical methods that are able to detect small amounts of these toxins, at the sub-cellular and molecular levels. But, even the newer detection methodology has its limitations. Therefore, early intervention by detection of these toxins remains a diagnostic and therapeutic need for preventing or reducing disease onset, assessing disease onset, as well as, disease progression.

Beryllium

Beryllium's unique properties make the metal an ideal choice for many industrial applications. It is lighter than aluminum, stiffer than steel, remains solid at high temperatures and can absorb large amounts of heat. Beryllium is used in the aerospace, computer, electronic and nuclear industries. Therefore, the use of Be in industry will continue to grow and the exposure to Be will continue to escalate due to this expanding use of the metal.

Be is not typically found in a human subject not exposed to environmental Be. When a subject is exposed to Be, search for Be in tissues or urine in suspected beryllium disease is often difficult due to inferior sensitivity of the methods employed. In one study, the clinical use of laser microprobe mass spectrometry (LAMMS) for measurement of Be was evaluated. It was found that this method detected the metal to a minimum concentration of 1 microM. The biological relevance of this concentration was evaluated. It was concluded that concentrations of Be in acute disease that exceed 1 microM were detectable by LAMMS. On the other hand, concentrations in chronic processes are below the detection limits of LAMMS. Therefore, new methods are needed to detect lower levels of Be found in a subject.

Approximately, one million American workers have been exposed to the metal beryllium and 1-16% of exposed individuals are at risk to develop chronic beryllium disease (CBD). In comparison to other human lung diseases such as sarcoidosis and hypersensitivity pneumonitis, CBD is a human granulomatous lung disease for which the causative antigen, beryllium (Be), is known. At-risk individuals include workers in defense, aerospace and airline, nuclear weapons, ceramics, computer, automotive, dental, electronics, alloy manufacturing, foundries and metal reclamation industries. While occupational exposures represent the major source of exposure resulting in illness, environmentally induced sensitization and disease due to non-occupational exposures continue to occur, but with unknown frequency. Beryllium is thought to cause injury to the lung, skin, and other organs through direct chemical toxic effects and through its ability to induce antigen-specific stimulation of cell-mediated immunity (CMI), however, the amount of Be-exposure necessary to induce and elicit these host responses remains unknown.

Therefore, more reliable tests with increased sensitivity are needed to assess disease onset and progression and target disease conditions for therapeutic treatment. In addition, more sensitive methods for detecting the progression of Be disease are needed.

### SUMMARY

Embodiments of the present invention provide for methods and compositions using Beryllium (Be), such as Be macromolecular complexes (eg.¹⁰Beryllium (¹⁰Be) macromolecular ligands or ⁷Be macromolecular ligands). In accordance with these embodiments, ¹⁰Be macromolecular ligands may be of use to detect and/or identify macromolecules such as receptors that bind such macromolecular complexes. In another embodiment, ¹⁰Be macromolecular ligands including a therapeutic agent may be of use to target a particular tissue or cellular population based on a receptor that binds such macromolecular complexes. In accordance with this embodiment, the delivery of one or more therapeutic agent may be delivered and/or monitored.

In another particular embodiment, ¹⁰Be complexes may be of use to identify metabolic pathways involved in various disease states. In accordance with this embodiment, ¹⁰Be complexes may be generated that target a specific metabolic enzyme or other molecule reflective of disease progression; to provide quantitative analysis of minute quantities of ¹⁰Be complexes; and/or to identify sub-cellular compartments, cells and/or tissues in which ¹⁰Be complexes become localized.

In one particular embodiment, Be complexed to a macromolecule may be used in the Beryllium Lymphocyte Proliferation Test (BeLPT) to assess exposure to Be and/or progression of Be disease in a sample from a subject.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1. illustrates an exemplary histogram of (a) TUNEL (assay for DNA strand break) positive nuclear staining of CBD BAL macrophages after exposure to 100 μM BeSO₄ and (b) nuclear fragmentation in CBD BAL macrophages exposed to Be-ferritin containing 270 picomoles of Be. (c) The percent (mean %±SEM) of CBD BAL cells (n=8) with TUNEL positive nuclei (black) or with fragmented nuclei (open) after exposure to 100 μM BeSO₄. *p<0.05 versus the corresponding unstimulated control.

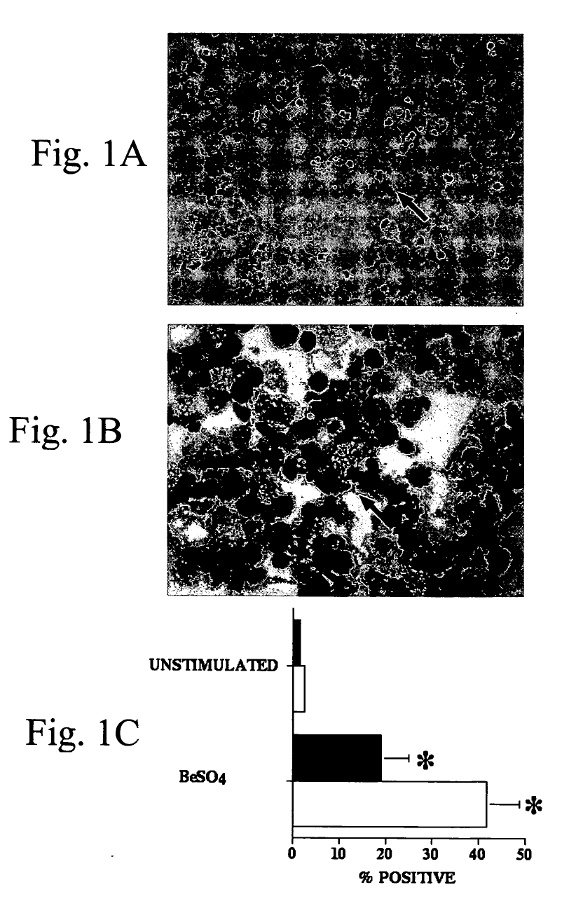

FIG. 2. represents an example of a TUNEL study comparing BeSO₄ stimulated and unstimulated control cells.

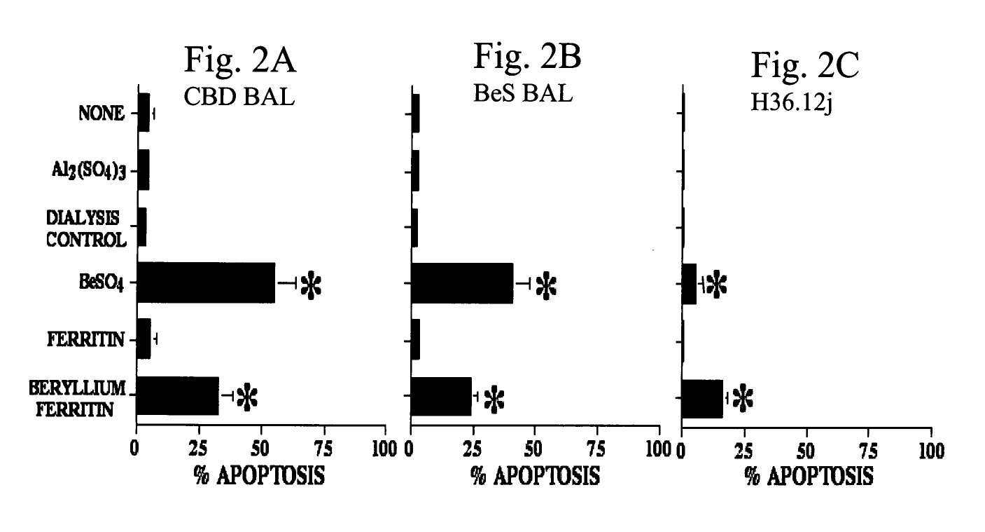

FIG. 3. represents an exemplary experiment utilizing a Be complex. The percent (mean %±SEM) of (A) CBD (Chronic Beryllium disease) BAL (n=5), (B) BeS BAL (n=15) and (C) H36.12j cells (n=5) with nuclear fragmentation that were unstimulated or exposed for 24 h to 100 μM Al₂(SO₄)₃, 50 μl of the “dialysis control,” 100 μM BeSO₄, 50 μg of ferritin alone or 50 μg of Be-ferritin containing 270 picomoles of Be.*p<0.05 versus the unstimulated controls.

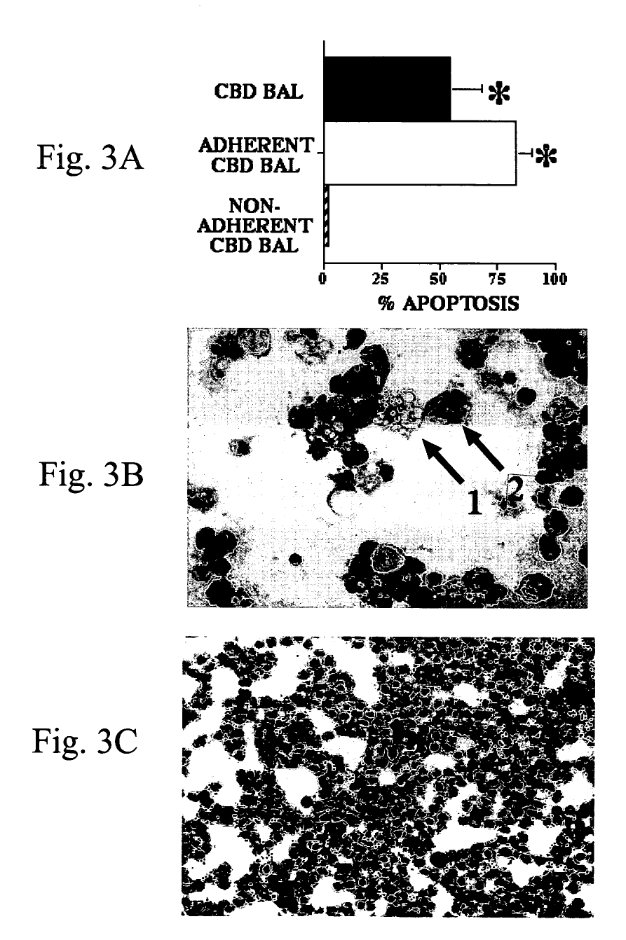

### DESCRIPTION OF ILLUSTRATIVE EMBODIMENTS

All documents, or portions of documents, cited in this application, including but not limited to patents, patent applications, articles, books, and treatises, are hereby expressly incorporated by reference in their entirety.

Definitions

As used herein, “a” or “an” may mean one or more than one of an item.

As used herein, the terms “and” and “or” may be used to mean either the conjunctive or disjunctive. That is, both terms should be understood as equivalent to “and/or” unless otherwise stated.

The use of the term “adduct” and/or “ligand” is encompassed within the scope of the term “complex” and illustrates exemplary embodiments of the claimed “complexes”.

A therapeutic agent is an atom, molecule, or compound that is useful in the treatment of a disease. Examples of therapeutic agents include antibodies, antibody fragments, drugs, toxins, enzymes, nucleases, hormones, immunomodulators, antisense oligonucleotides, small interfering RNA (siRNA), aptamers, and chelators. Other exemplary therapeutic agents and methods of use are disclosed in U.S. Patent Publication Nos. 20050002945, 20040018557, 20030148409 and 20050014207, each incorporated herein by reference.

A diagnostic agent is an atom, molecule, or compound that is useful in diagnosing a disease, either by in vitro or in vivo tests. Useful diagnostic agents include, but are not limited to, radioisotopes, dyes (such as with the biotin-streptavidin complex), contrast agents, fluorescent compounds or molecules, and enhancing agents (e.g., paramagnetic ions) for magnetic resonance imaging (MRI).

### DESCRIPTION

In the following sections, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods or components have not been included in the description.

In some embodiments, methods of use of Be complexes may include detection, diagnosis and/or treatment of a disease or other medical condition. Such conditions may include, but are not limited to, cancer, hyperplasia, diabetic retinopathy, macular degeneration, inflammatory bowel disease, beryllium disease, ulcerative colitis, rheumatoid arthritis, diabetes, sarcoidosis, asthma, edema, pulmonary hypertension, psoriasis, corneal graft rejection, neovascular glaucoma, myocardial angiogenesis, plaque neovascularization, restenosis, neointima formation after vascular trauma, telangiectasia, hemophiliac joints, angiofibroma, fibrosis associated with chronic inflammation, lung fibrosis, amyloidosis, Alzheimer's disease, organ transplant rejection, deep venous thrombosis or wound granulation.

In one embodiment, Be complexes of the present invention may be include Be complexed to a macromolecule for therapeutic and/or diagnostic purposes. It is contemplated herein that Be such as ¹⁰Be can be used in any of the disclosed methods herein to make adducts or complexes with any variety of molecules, inorganic (such as salts) or organic simple or complex compounds, viruses and prions that could be linked to Be. For example, the chemical link may include chemically binding Be to one of the aforementioned molecules via a covalent bond or non-covalent bond.

In certain embodiments, the Be complexes may be of use for therapeutic diagnosis and/or treatment of cancer. It is anticipated that any type of cancer and/or any type of tumor antigen may be targeted for diagnostic and/or therapeutic purposes. Exemplary types of tumors that may be targeted include, but are not limited to, acute lymphocytic leukemia, acute myelogenous leukemia, biliary cancer, breast cancer, cervical cancer, chronic lymphocytic leukemia, chronic myelogenous leukemia, colorectal cancer, endometrial cancer, esophageal cancer, gastric cancer, head and neck cancers, Hodgkin's lymphoma, lung cancer, medullary thyroid cancer, non-Hodgkin's lymphoma, multiple myeloma, renal cancer, ovarian cancer, pancreatic cancer, melanoma, liver cancer, prostate cancer, glial and other brain and spinal cord tumors, and urinary bladder cancer.

In other embodiments, the Be complexes disclosed herein may be of use to detect and/or treat infection by a pathogenic organisms, such as bacteria, viruses, fungi, unicellular parasites or macromolecules associated with a pathogenic organism. Exemplary fungi that may be treated include but are not limited to Cryptococcus neoformans, Histoplasma capsulatum, Blastomyces dermatitidis, Candida albican or combination thereof. Exemplary viruses include but are not limited to human immunodeficiency virus (HIV), herpes virus, cytomegalovirus, influenza virus, human papilloma virus, hepatitis B virus, hepatitis C virus, Sendai virus, feline leukemia virus, Reo virus, polio virus, human serum parvo-like virus, simian virus 40, respiratory syncytial virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, or combination thereof. Exemplary bacteria include but are not limited to Streptococcus agalactiae, Legionella pneumophilia, Streptococcus pyogenes, Escherichia coli, Neisseria gonorrhoeae, Neisseria meningitidis, Pneumococcus spp., Mycobacterium leprae, Brucella abortus, Pseudomonas aeruginosa ,Mycobacterium tuberculosis, Mycoplasma pneumonia or combination thereof Exemplary parasites include but are not limited to Giardia lamblia, Giardia spp., Toxoplasma gondii, Cryptospordium spp., Leishmania spp., Trypanosoma evansi, Dientamoeba fragilis, Trichomonas vaginalis, Plasmodiumfalciparum, Isospora spp., Toxoplasma spp. Enterocytozoon spp., Pneumocystis spp., Balantidium spp or combination thereof.

In one embodiment, one or more protein or peptide therapeutic or diagnostic agents may be attached to or incorporated into a Be complex for diagnostic or therapeutic application to a subject. Examples of these agents include but are not limited to a bacterial toxin, a plant toxin, ricin, abrin, a ribonuclease (RNase), DNase I, Staphylococcal enterotoxin-A, pokeweed antiviral protein, gelonin, diphtherin toxin, Pseudomonas exotoxin, Pseudomonas endotoxin, Ranpirnase (Rap), Rap (N69Q), PE38, dgA, DT390, PLC, tPA, a cytokine, a growth factor, a soluble receptor component, surfactant protein D, a clot-dissolving agent, an enzyme, anti-angiogenic agent, an antibody, an antibody fragment or combination thereof.

Beryllium

Beryllium, atomic number of four, an atomic weight of 9.013 and a charge number z=2, heads the Group ELA, alkaline earth elements of the periodic table. The chemical properties of Be are due to this high density of charge. In the other group DA. elements z/r ranges from 0.65 to 1.35, and because of its small size Be cannot expand its coordination number above 4. Therefore, in complex compounds Be2⁺ accepts two extra electrons and forms tetrahedral structures, principally with oxygen, due to its high z/r. In aqueous solution, Be ions are hydrated and beryllium may readily form hydroxides. At pH>6, highly reactive Be(OH)₂ dominates and is a likely chemical form of Be that may interacts with a protein, peptide or other macromolecule.

Previous studies show that the principal molecule bound by the exposure of hepatocytes to ⁷BeCl₂ (⁷Be T1/2=53 d) was the ubiquitous iron transport and storage protein ferritin. Beryllium, with an atomic weight of 9.013 and a charge number z =2, has a small ionic radius, r=0.31 nm. The ratio of the charge number to the radius is large, z/r=6.45 and the chemical properties of Be are due to this high density of charge. Be can accept two extra electrons and is capable of forming complex compounds. One compound capable of binding Be is ferritin. Another embodiment of the invention may include a Be complex composition where beryllium in its natural or isotopic form may be attached to a macromolecule known to accept and or associate with any other divalent metal ions. It is contemplated herein that any compound capable of binding a divalent cation can also bind to Be.

Beryllium occurs in several chemical forms including:

⁹Beryllium—is the stable, non-radioactive, naturally occurring element with a limit of detection of about 10 μM ⁹Be by atomic absorption and conventional mass spectroscopy. ⁹Be levels must be relatively concentrated since conventional mass spectroscopy is limited to the detection of elements with an atomic mass>15, the atomic mass of ⁹Be=9.32.

⁷Beryllium—is a short half-life radionuclide of beryllium, T_{1/2}=53 d, that has been used in previous studies. It can be measured using a gamma counter with a limit of detection of about 10 μM ⁷Be.

¹⁰Beryllium—is a long-lived radionuclide, T_{1/2}=2.3 million years that can be detected at 1×10¹⁸ M levels by AMS

In one aspect of this invention, the present invention elicits the use of Be in a form that requires extremely low levels of the divalent cation that have been shown to be non-toxic.

Ferritin

Ferritin is a high molecular weight protein (MW>400,000 Daltons) composed of 24 subunits [3]. Each subunit is composed of four a helices that form parallel cylinders creating an external protein shell and a heavily phosphorylated internal core region that binds approximately 4,500 ferric atoms in a crystalline inorganic complex. It has been demonstrated that using sulfosalicylic acid Be metal can be removed from a Be-ferritin adduct. This demonstrated that Be tightly binds to the ferritin subunit core region, not to the external protein shell, with no displacement of iron from the core. The tightness of Be binding occurs through the formation of covalent bonds between Be2* and the local phosphate groups inside the subunit core region. In one example, ferritin was capable of binding 800 gram atoms of Be, suggesting that a single ferritin molecule forms a chemical adduct with small numbers of Be atoms.

It has also been demonstrated that minute levels Be complexed to the macromolecule ferritin (“Be-ferritin adduct”) are capable of delivering Be to lung macrophages (See Example 1).

In one embodiment, 10BAPs (¹⁰Be-Adduct Probes) can be designed to trace intracellular pathways at very low levels of the radioisotope ¹⁰Be. In another example, 10BAPs (¹⁰Be-Adduct Probes) can be designed for use as tool to detect, identify and define heretofore unknown sub-cellular interactions of the labeled adduct with a variety of chemicals, proteins and other molecules and macromolecules, including viruses and prions.

Be occurs in nature in low abundance and is not normally found inside the human body such as human tissues, organs, fluids or body cavities. ¹⁰Be in particular is an isotope of stable ⁹Be and does not occur in biologic systems. ¹⁰Be is not found inside human cells associated with cell organelles, proteins, fluids or macromolecules. ⁹Be is only found associated with human tissues and cells in individuals who are exposed to Be, usually by occupational exposure, or who have BeS (beryllium sensitized) or disease. Thus, the exposure of tissues and cells to experimentally developed 10BAPs of the present invention results in the presence of the ¹⁰Be-Adduct in those human tissues and cells unequivocally derived from a 10BAP (see Example 2) administered to a subject and/or a sample.

Beryllium Lymphocyte Proliferation Test (BeLPT)

The standard assay for documenting the presence of a beryllium-specific immune response in blood is the beryllium lymphocyte proliferation test (BeLPT) (Rossman et al., Ann Intern Med 1988, 108:687-93; Mroz et al., J Allergy Clin Immunol 1991, 88:54-60, the entire text of each of which is incorporated herein by reference). Due to the sensitivity of this assay, it has been used for screening and diagnosis of beryllium sensitization in the workplace and is a required component of the US Department of Energy CBD prevention program (Chronic beryllium disease prevention program. Office of Environment, Safety and Health, Department of Energy. Final rule. Fed Regist 1999;64:68854-914). However, it has been criticized due to variability in test results and lack of sensitivity. In addition, the BeLPT is not capable of distinguishing between BeS and CBD, which currently requires invasive tests such as bronchoscopy with bronchoalveolar lavage (BAL) and lung biopsy to confirm progression to CBD. Certain embodiments of the present invention concern methods of use of Be-protein complexes, such as Be-ferritin, to perform BeLPT assays with greater sensitivity and/or accuracy.

Research has established three clinically distinct groups of Be-exposed individuals: (I) Beryllium-exposure, non-diseased, are individuals with documented exposure to Be, but who display no overt clinical changes in pulmonary function or immunologic status. (2) Beryllium sensitized (BeS) are individuals with documented Be exposure, no clinical symptoms or changes in pulmonary function and normal lung histology, but whose blood lymphocytes show a Be-specific cell mediated immunologic response upon in vitro Be-stimulation. Although the majority of these patients are healthy initially, approximately 11% progress to CBD per year. (3) CBD, the presence of non-caseating granulomas in the lungs, evidenced either radiologically or by biopsy, accompanied by marked changes in the blood and BAL cell immunologic response in vitro to Be-stimulation.

The presence, or absence, of a CMI response in Be-exposed subjects is used clinically to establish disease progression from Be-exposure, with no CMI responses in either the PBMC or BAL cell compartments, to BeS with a positive CMI response in the PBMCs but not the BAL cell compartment and then to CBD with a positive CMI response in both the PBMCs and the BAL cell compartments. CMI is indicative of the presence, either in the PBMC or BAL cell compartments, of Be-specific, CD4⁺ effector-memory T lymphocytes. When either PBMC or BAL cells that contain these Be-specific T cells, are placed in culture and exposed to graded concentrations of Be-salts, they proliferate (see BeLPT discussed previously). The BeLPT is used as the standard clinical diagnostic tool to determine the CMI status of an individual with a history of Be-exposure. Clinical decisions to treat patients who are progressing from BeS to CBD with an increased pulmonary dysfunction and demonstrable granulomatous lung disease, are based in part on a positive PBMC and BAL BeLPT.

Example BeLPT Assay

A positive CMI response in either the PBMC or BAL cell compartments is established by determining the stimulation index (SI) after in vitro Be-exposure. The SI is determined from the ratio of the cpm in the treated cultures to the cpm in the unstimulated control cultures. Thus, the ratio of the cpm in the unstimulated controls to itself would=1. If the cpm in the treated cultures are higher than the cpm in the unstimulated controls then the SI>1. It has been demonstrated that when a treated culture's SI greater than or equal to 2.5, then a significant amount of Be-specific CD4⁺ T cell proliferation has occurred in those wells. In one example, a treatment group can consist of three wells each containing approximately 2×10⁸ PBMC or BAL cells per well, that are unstimulated or stimulated with 100 μM, 10 μM or 1 μM BeSO₄. BeSO₄ is chosen for this assay because the sulfate group renders Be soluble in aqueous solutions without major effects on the pH of the culture medium (normally=pH 7.2 to 7.4). As controls for the addition of Be-metal salts, in some instances equal amounts of Al₂(SO₄)₃ aluminum sulfate, are added=the metal-salt control.

Added to separate sets of wells were control substances that positively stimulated T cell proliferation: 1) 1 μg/ml phytohemagglutanin (PHA, Sigma) which is a non-specific T cell mitogen that triggers the proliferation of all T cells in culture, SI>2.5. 2) Candida albicans antigen: a culture filtrate protein antigen from the fungus C. albicans that depends on presentation by major histocompatibility (MHC) Class n moleculesm SI>2.5. 3) Trychophyton terrestrie antigen: a culture filtrate protein antigen from the fungus T.lerrestrie that depends on presentation by MHC Class II molecules, SI>2.5. 4)Tetanus toxoid: a formaldehyde toxoid preparation of tetanus toxin produced by Clostridium tetani that that depends on presentation by MHC Class II molecules, SI>2.5.

The rationale for using this number of positive controls is that any individual patient may or may not have sufficient levels of CD4+ memory T cells directed against all four MHC class II dependent antigens at any given time, however, the majority of patients have a CMI memory response to at least one of these antigens. All patient cells should proliferate in response to PHA-stimulation indicating the general health of the T cell population.

Sets of plates treated in this manner are incubated at 37° C. in an humidified atmosphere containing 5% CO2 and on days 4, 5 and 6 after treatment the cells are ³HTdR-pulse labeled and the cpm determined for each well. Means are calculated for triplicate samples and the SI ratios determined as described. Normally, all of the SI data are clinically reported, however, for publication purposes we normally report the “peak SI” selected from the group of plates and Be-stimulated amounts. As an example, the PBMC and BAL cell BeLPT SI for BeS (n=19) and CBD (n=8) subjects are shown in Table 1.

TABLE 1
The PBMC and BAL cell BeLPT stimulation index (SI).
Median SI, (minimum; maximum),

| | BeS (n = 19) | CBD (n = 8) |
|---|---|---|
| PBMC BeLPT SI | 3.4, (1.1; 30)* | 9.2; (1.1; 17)* |
| BAL BeLPT SI | 1.9, (1.2, 46) | 95, (1.2, 190)* |

*p < 0.05, Tukey Kramer test versus an unstimulated control SI > 2.5.

In one embodiment of the present invention, a Be complex may be used in a beryllium lymphocyte proliferation test (BeLPT) on a sample from a subject. In accordance with this embodiment, a Be complex may be used to induce T-cell proliferation on a lymphocyte cell sample to diagnose the presence of a beryllium-specific immune response in blood. In another embodiment, a Be complex can include a beryllium complex of Be and a macromolecule where beryllium can be ⁹Be, ¹⁰Be, ⁷Be or combination thereof. Example macromolecules can include ferritin, lactoferrin, transferrin, metallothionein or ceruloplasmin. In one embodiment, the BeLPT test is performed at a Be concentration that is at least an order of magnitude lower than the concentrations of inorganic salts of beryllium used to perform current BeLPT assays. In another embodiment, the BeLPT test is performed at a Be concentration that is at least three orders of magnitude lower than the concentrations of inorganic salts of beryllium used to perform BeLPT assays. In one particular embodiment, the concentration of the Be-complex in the BeLPT assay is less than 1 micromolar. In another particular embodiment, the concentration of the Be-complex in the BeLPT assay is less than 1 nanomolar.

AMS:

AMS is a technique for determining isotope ratios with very high sensitivity. Relative to other techniques that measure the isotope ratios of abundant stable isotopes such as isotope ratio mass spectrometry (IRMS), AMS measures the ratio of a rarer radioisotope relative to a more abundant stable isotope of the same element. AMS for example measures ¹⁴C/¹³C, ¹⁰Be/9Be, or ³H/¹H. AMS provides the ability to quantitatively trace radioisotopes such as ¹⁰Be to 1×10⁻¹⁸ moles and offers the possibility to measure isotope labeled biologies at levels of physiological relevance in vivo. The Lawrence Livermore National Laboratory, Livermore, Calif. houses the National Resource for Biomedica) Accelerator Mass Spectroscopy (AMS: The National Resource for Biomedical AMS website. These instruments are currently being developed for more general us in any laboratory.

AMS is a type of mass spectrometer that uses a Van Der Graaff electrostatic accelerator to accelerate negative ions produced in a SIMS of FAB type ion source at MV potentials. The ions are stripped of elections at the terminal of the accelerator to destroy molecular isobars of the isotope being analyzed and then reaccelerated as a positive ion. The high-energy positive atomic ions are then separated using magnets and velocity filters followed by identification of the total energy and energy loss of each ion as it is individually counted in the detector. By combining momentum analysis, velocity analysis, energy analysis and energy loss analysis, AMS can detect ions and measure isotope ratios as low as 1:1′^{N} to 1:10″¹⁸ with precision of 0.25 to 2%. AMS is a powerful new technique for measuring radioisotopes, which allows such studies to be conducted using human relevant exposure situations and with less compound that was possible with other techniques. It also allows studies to be conducted directly in humans that were not possible previously by virtue of the ability of AMS to measure isotope-labeled agents well below toxicity or natural environmental levels.

Accelerator Mass Spectroscopy, AMS, can measure the ratio of ¹⁰Be/9Be at ¹⁰Be levels of 1×10⁻¹⁸ M. Be is not normally present in human tissues or fluids and AMS has been adapted to overcome the limitations of detection sensitivity of long-lived radionuclides that can not be analyzed with decay counting or conventional mass spectrometry. AMS is extremely sensitive for counting atomic nuclei, is able to detect approximately 1×10⁻¹⁵ to 1×10⁻¹⁸ M Be, in sample sizes that are a thousand-fold smaller as compared to decay counting.

LLNL is a National Laboratory organized to facilitate multidisciplinary sharing of facilities and equipment among its staff and collaborators. Resource facilities at LLNL also include laboratories for sample preparation, chemical separations, physical analysis and cell culture. The 10 MV AMS instrument is contained in a 7,000 square foot building located in the northwest corner of LLNL. The AMS spectrometer is built around a High Voltage Engineering Corporation” model FN tandem accelerator capable of terminal voltages up to 10 MV. The AMS spectrometer consists of a cesium sputter source, low-energy injection beam line, a high energy mass spectrometer and a multi-anode ionization detector for energy and energy-loss measurements. Other AMS facilities currently exist.

It is contemplated herein, that any method or instrument to detect the presence of any form of beryllium now or in the future is encompassed within the present invention. For example, it is contemplated that a sample or subject screened for the presence or absence of a macromolecule of the present invention using a Be-complex such as Be-protein, Be-protein-therapeutic agent (Be-ferritin, Be-receptor ligand-therapeutic agent) may be screened using a fluorescent detector, a radiation detector, a spectrophotometer, mass spectrometer, an AMS spectrometer and the like.

Be-Ferritin

In one exemplary complex, Beryllium (Be) forms adducts with ferritin (Be-ferritin) (see Example 1). In a previous study, it was shown there was an increased frequency of apoptotic bronchoalveolar lavage (BAL) macrophages from CBD subjects after exposure to 100 μM BeSO₄ (50%±6%, mean±SEM, p<0.05 versus none) and to a ferritin adduct containing 270 picomoles of Be (40%±2%). Increased Be-ferritin induced apoptosis was observed in BAL macrophages from subjects with Be-sensitization (BeS=25%±3%) and in the H36.12j hybrid macrophage cell line (15%±2%). Be-ferritin stimulated Be-specific CBD BAL T cell proliferation concentrations 5-6 logs lower than the amounts of BeSO₄ needed to induce comparable results. Thus, lung macrophages are capable of taking up Be-ferritin and delivering physiologically relevant levels of Be that promote Be-antigen presentation and macrophage apoptosis. These results support the use of Be complexed to macromolecules as a potential potent diagnostic and/or delivery tool.

Pharmaceutical Compositions

In one embodiment, it is contemplated that any Be complex disclosed herein may include one or more therapeutic agent. In accordance with this embodiment, a therapeutic agent may include, but is not limited to one or more of a drug, a toxin, a prodrug, a toxin, an enzyme, a protease, an enzyme-inhibitor, a nuclease, a hormone, a hormone antagonist, an anti-inflammatory agent, an anti-cancer agent, an immunomodulator, an oligonucleotide, a boron compound, a photoactive agent or combinations thereof.

Other therapeutic agents contemplated for use herein may include but are not limited to one or more of the following: azacytidine, bleomycin, busulfan, camptothecin, carmustine, celebrex, chlorambucil, cisplatin, irinotecan (CPT-11), SN-38, carboplatin, cladribine, cyclophosphamide, cytarabine, daunomycin glucuronide, daunorubicin, dexamethasone, diethylstilbestrol, doxorubicin, epirubicin glucuronide, estramustine, etoposide, etoposide glucuronide, etoposide phosphate, floxuridine (FUdR), 3′,5′-O-dioleoyl-FudR (FUdR-dO), fludarabine, flutamide, fluorouracil, gemcitabine, hydroxyprogesterone caproate, hydroxyurea, idarubicin, L-asparaginase, leucovorin, lomustine, mechlorethamine, medroprogesterone acetate, megestrol acetate, melphalan, mercaptopurine, 6-mercaptopurine, methotrexate, mitomycin, mitotane, phenyl butyrate, prednisone, paclitaxel, pentostatin, tamoxifen, taxanes, taxol, testosterone propionate, thalidomide, thioguanine, thiotepa, teniposide, topotecan, uracil mustard, vinblastine, vinorelbine, vincristine, ricin, abrin, ribonuclease, ribonuclease, onconase, rapLR1, DNase I, Staphylococcal enterotoxin-A, pokeweed antiviral protein, gelonin, diphtheria toxin, Pseudomonas exotoxin, Pseudomonas endotoxin, nitrogen mustard, ethyleneimine derivative, alkyl sulfonate, nitrosourea, triazene, folic acid analog, anthracycline, COX-2 inhibitor, pyrimidine analog, purine analog, antibiotic, epipodophyllotoxin, platinum coordination complex, vinca alkaloid, substituted urea, methyl hydrazine derivative, adrenocortical suppressant, antagonist, endostatin, cytokine, interleukin, interferon, lymphokine, tumor necrosis factor, antisense oligonucleotide, interference RNA, and combinations thereof.

Therapeutic agents include but are not limited to, for example, chemotherapeutic drugs such as vinca alkaloids, anthracyclines, epidophyllotoxins, taxanes, antimetabolites, alkylating agents, antibiotics, antimitotics, antiangiogenic and proapoptotic agents, particularly doxorubicin, methotrexate, taxol, CPT-11, SN-38, camptothecans, and others from these and other classes of anticancer agents, and the like. Other cancer chemotherapeutic drugs include nitrogen mustards, alkyl sulfonates, nitrosoureas, triazenes, folic acid analogs, pyrimidine analogs, purine analogs, platinum coordination complexes, hormones, and the like. Suitable chemotherapeutic agents are described in Remington's Pharmaceutical compositions, 19th Ed. (Mack Publishing Co. 1995), and in Goodman and Gilman's the Pharmacological Basis of Therapautics, 7th Ed. (MacMillan Publishing Co. 1985), as well as revised editions of these publications. Other suitable chemotherapeutic agents, such as experimental drugs, are known to those of skill in the art, and may be conjugated to the bioactive assemblies described herein using methods that are known in the art.

Exemplary therapeutic peptides or proteins may include but is not limited to, for example: adrenocorticotropic hormone (ACTH); adrenocorticotropic hormone derivatives (e.g., ebiratide); angiotensin; angiotensin II; asparaginase; atrial natriuretic peptides; atrial sodium diuretic peptides; bacitracin; beta-endorphins; blood coagulation factors VII, VIII and IX; blood thymic factor (FTS); blood thymic factor derivatives (see U.S. Pat. No. 4,229,438); bombesin; bone morphogenic factor (BMP); bone morphogenic protein; bradykinin; caerulein; calcitonin gene related polypeptide (CGRP); calcitonins; CCK-8; cell growth factors (e.g., EGF; TGF-alpha; TGF-beta; PDGF; acidic FGF; basic FGF); cerulein; chemokines; cholecystokinin; cholecystokinin-8; cholecystokinin-pancreozymin (CCK-PZ); colistin; colony-stimulating factors (e.g. CSF; GCSF; GMCSF; MCSF); corticotropin-releasing factor (CRF); cytokines; desmopressin; dinorphin; dipeptide; dismutase; dynorphin; eledoisin; endorphins; endothelin; endothelin-antagonistic peptides (see European Patent Publication Nos. 436189; 457195 and 496452 and Japanese Patent Unexamined Publication Nos. 94692/1991 and 130299/1991); endotherins; enkephalins; enkephalin derivatives (see U.S. Pat. No. 4,277,394 and European Patent Publication No. 31567); epidermal growth factor (EGF); erythropoietin (EPO); follicle-stimulating hormone (FSH); gallanin; gastric inhibitory polypeptide; gastrin-releasing polypeptide (GRP); gastrins; G-CSF; glucagon; glutathione peroxidase; glutathio-peroxidase; glutaredoxin; gonadotropins (e.g., human chorionic gonadotrophin and alpha. and .beta. subunits thereof); gramicidin; gramicidines; growth factor (EGF); growth hormone-releasing factor (GRF); growth hormones; hormone releasing hormone (LHRH); human artrial natriuretic polypeptide (h-ANP); human placental lactogen; insulin; insulin-like growth factors (IGF-I; IGF-II); interferon; interferons (e.g., alpha- beta- and gamma-interferons); interleukins (e.g. 1; 2; 3; 4; 5; 6; 7; 8; 9; 10; 11 and 12); intestinal polypeptide (VIP); kallikrein; kyotorphin; luliberin; luteinizing hormone (LH); luteinizing hormone-releasing hormone (LH-RH); lysozyme chloride; melanocyte-stimulating hormone (MSH); melanophore stimulating hormone; mellitin; motilin; muramyl; muramyldipeptide; nerve growth factor (NGF); nerve nutrition factors (e.g. NT-3; NT-4; CNTF; GDNF; BDNF); neuropeptide Y; neurotensin; oxytocin; pancreastatin; pancreatic polypeptide; pancreozymin; parathyroid hormone (PTH); pentagastrin; polypeptide YY; pituitary adenyl cyclase-activating polypeptides (PACAPs); platelet-derived growth factor; polymixin B; prolactin; protein synthesis stimulating polypeptide; PTH-related protein; relaxin; renin; secretin; serum thymic factor; somatomedins; somatostatins derivatives (Sandostatin; see U.S. Pat. Nos. 4,087,390; 4,093,574; 4,100,117 and 4,253,998); substance P; superoxide dismutase; taftsin; tetragastrin; thrombopoietin (TPO); thymic humoral factor (THF); thymopoietin; thymosin; thymostimulin; thyroid hormone releasing hormone; thyroid-stimulating hormone (TSH); thyrotropin releasing hormone TRH); trypsin ;thyroidoxin; tuftsin; tumor growth factor (TGF-alpha); tumor necrosis factor (TNF); tyrocidin; urogastrone; urokinase; vasoactive intestinal polypeptide; vasopressins, and functional equivalents of such polypeptides.

A suitable peptide containing a detectable label (e.g., a fluorescent molecule), or a cytotoxic agent, (e.g., a radioiodine), can be covalently, non-covalently, or otherwise associated with any Be complex disclosed herein. For example, a therapeutically useful conjugate can be obtained by incorporating a photoactive agent or dye onto the bioactive assemblies. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See :van den Bergh, Chem. Britain (1986), 22:430. Moreover, monoclonal antibodies have been coupled with photoactivated dyes for achieving phototherapy. See Mew et al., J. Immunol. (1983),130:1473.

Be Complex Administration

Various embodiments of the claimed methods and/or compositions may concern one or more Be-complex to be administered to a subject. Administration may occur by any route known in the art, including but not limited to oral, nasal, buccal, inhalational, rectal, vaginal, topical, orthotopic, intradermal, subcutaneous, intramuscular, intraperitoneal, intraarterial, intrathecal or intravenous injection. In one embodiment, a traceable Be complex having a therapeutic agent attached to the complex may be of use to track the delivery of the agent to a specified cell, tissue or organ.

Methods for chemically modifying peptides to render them less susceptible to degradation by endogenous proteases or more absorbable through the alimentary tract are well known (see, for example, Blondelle et al., 1995, Biophys. J. 69:604-11; Ecker and Crooke, 1995, Biotechnology 13:351-69). Methods for preparing libraries of peptide analogs, such as peptides containing D-amino acids; peptidomimetics consisting of organic molecules that mimic the structure of a peptide; or peptoids such as vinylogous peptoids, have also been described and may be used to construct peptide based bioactive assemblies suitable for oral administration to a subject. Peptide stabilization may also occur by substitution of D-amino acids for naturally occurring L-amino acids, particularly at locations where endopeptidases are known to act.

In certain embodiments, the standard peptide bond linkage may be replaced by one or more alternative linking groups, such as CH₂—NH, CH₂—S, CH₂—CH₂, CH═CH, CO—CH₂, CHOH—CH₂ and the like. Methods for preparing peptide mimetics are well known in the art. (for example Holladay et al., 1983, Tetrahedron Lett. 24:4401-04; and Almquiest et al., 1980, J. Med. Chem. 23:1392-98). Peptide mimetics may exhibit enhanced stability and/or absorption in vivo compared to their peptide analogs.

In still other embodiments, peptides may be modified for oral or inhalafional administration by conjugation to certain proteins.

It is contemplated that any Be complex disclosed herein may be delivered encapsulated by methods known in the art such as within a gel, a microbead, a microparticle, a matrix formulation or the like. It is also contemplated that any Be complex disclosed herein and administered to a subject or a sample may be administered as a rapid release formulation or a time released formulation.

Proteins and Peptides

A variety of polypeptides or proteins may be used within the scope of the claimed methods and compositions. In certain embodiments, the proteins can include proteins such as ferritin or antibodies or fragments of antibodies containing an antigen-binding site. As used herein, a protein, polypeptide or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide” are used interchangeably herein. Accordingly, the term “protein or peptide” encompasses amino acid sequences comprising at least one of the 20 common amino acids found in naturally occurring proteins, or at least one modified or unusual amino acid.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid, including but not limited to those shown below. Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed and may be found at computerized databases known to those of ordinary skill in the art. One such database is the National Center for Biotechnology Information's Genbank and GenPept databases (www.ncbi.nlm.nih.gov/). The coding regions for known genes may be amplified and/or expressed using the techniques disclosed herein or as would be know to those of ordinary skill in the art. Alternatively, various commercial preparations of proteins, polypeptides, and peptides are known to those of skill in the art.

Fusion Proteins

Various embodiments may concern fusion proteins. These molecules generally have all or a substantial portion of a peptide, linked at the N- or C-terminus, to all or a portion of a second polypeptide or protein. Methods of generating fusion proteins are well known to those of skill in the art. Such proteins may be produced, for example, by chemical attachment using bifunctional cross-linking reagents, by de novo synthesis of the complete fusion protein, or by attachment of a DNA sequence encoding a first protein or peptide to a DNA sequence encoding a second peptide or protein, followed by expression of the intact fusion protein.

Synthetic Peptides

Proteins or peptides may be synthesized, in whole or in part, in solution or on a solid support in accordance with conventional techniques. Various automatic synthesizers are commercially available and can be used in accordance with known protocols. See, for example, Stewart and Young, (1984, Solid Phase Peptide Synthesis, 2d. ed., Pierce Chemical Co.); Tam et al., (1983, J Am. Chem. Soc., 105:6442); Merrifield, (1986, Science, 232: 341-347); and Barany and Merrifield (1979, The Peptides, Gross and Meienhofer, eds., Academic Press, New York, pp. 1-284). Short peptide sequences, usually from about 6 up to about 35 to 50 amino acids, can be readily synthesized by such methods. Alternatively, recombinant DNA technology may be employed wherein a nucleotide sequence which encodes a peptide of interest is inserted into an expression vector, transformed or transfected into an appropriate host cell, and cultivated under conditions suitable for expression.

Antibodies

Various embodiments may concern antibodies for a target. The term “antibody” is used herein to refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Harlowe and Lane, 1988, Antibodies: A Laboratory Manual, Cold Spring Harbor Laboratory). Antibodies of use may also be commercially obtained from a wide variety of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and are available for use in the claimed methods and compositions. (See, for example, U.S. Pat. Nos. 7,060,802; 7,056,509; 7,049,060).

Production of Antibody Fragments

Some embodiments of the claimed methods and/or compositions may concern antibody fragments. Such antibody fragments may be obtained by pepsin or papain digestion of whole antibodies by conventional methods. For example, antibody fragments may be produced by enzymatic cleavage of antibodies with pepsin to provide F(ab′)₂ fragments. This fragment may be further cleaved using a thiol reducing agent and, optionally, followed by a blocking group for the sulfhydryl groups resulting from cleavage of disulfide linkages, to produce Fab′ monovalent fragments. Alternatively, an enzymatic cleavage using papain n produces two monovalent Fab fragments and an Fc fragment. Exemplary methods for producing antibody fragments are disclosed in U.S. Pat. No. 4,036,945; and U.S. Pat. No. 4,331,647).

It is contemplated herein that any antibody of antibody fragment used in a Be-complex may be a chimeric, human or humanized antibody generated by means known in the art.

Methods of Disease Tissue Detection, Diagnosis and Imaging

Protein-Based In Vitro Diagnosis

The present invention contemplates the use of Be complexes to screen biological samples in vitro and/or in vivo for the presence of condition- and/or disease-associated macromolecules. In exemplary assays, a Be complex can detect or adhere to a macromolecule such as a protein, peptide, nucleic acid, an antibody, fusion protein, or fragment thereof may be utilized in liquid phase or bound to a solid-phase carrier, as described below. The skilled artisan will realize that a wide variety of techniques are known for determining levels of expression of a particular gene and any such known method, such as immunoassay, RT-PCR, mRNA purification and/or cDNA preparation followed by hybridization to a gene expression assay chip may be utilized to determine levels of expression in individual subjects and/or tissues. Exemplary in vitro assays of use include RIA, ELISA, sandwich ELISA, Western blot, slot blot, dot blot, and the like. Although such techniques were developed using intact antibodies, bioactive assemblies that incorporate antibodies, antibody fragments or other binding moieties may be used. It is contemplated herein that any disclosed Be complex may be attached to a chip, slide and/or any known array device in the art for monitoring or detection thereof.

Be complexes can be additionally labeled with any appropriate marker moiety, for example, a radioisotope, an enzyme, a fluorescent label, a dye, a chromogen, a chemiluminescent label, a bioluminescent label or a paramagnetic label. The marker moiety may be a radioisotope that is detected by such means as the use of a gamma counter or a beta-scintillation counter or by autoradiography.

In Vivo Diagnosis

Methods of diagnostic imaging with labeled macromolecules are well-known. For example, in the technique of immunoscintigraphy, ligands or antibodies are labeled with a gamma-emitting radioisotope and introduced into a patient. A gamma camera is used to detect the location and distribution of gamma-emitting radioisotopes.

The radiation dose delivered to the patient is maintained at as low a level as possible through the choice of isotope for the best combination for a primary or secondary detection of a complex (radiolabelled Be, may be the primary agent detected) of minimum half-life, minimum retention in the body, and minimum quantity of isotope which will permit detection and accurate measurement.

Imaging Agents for Secondary Detection of a Be Complex and Radioisotopes

Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, carbon¹⁴, chromium⁵¹, chlorine³⁶, cobalt⁵⁷, cobalt⁵⁸, copper⁶², copper⁶⁴, copper⁶⁷, Eu¹⁵², fluorine¹⁸, gallium⁶⁷, gallium⁶⁸, hydrogen³, iodine¹²³, iodine¹²⁴, iodine¹²⁵, iodine¹³¹, indium¹¹¹, iron⁵², iron⁵⁹, lutetium¹⁷⁷, phosphorus32, phosphorus³³, rhenium¹⁸⁶, rhenium¹⁸⁸, Sc¹⁴⁷, selenium⁷⁵, silver¹¹¹, sulphur³⁵, technetium^{94m}, technetium^{99m}, yttrium⁸⁶ and yttrium⁹⁰, and zirconium⁸⁹. I¹²⁵ is often being preferred for use in certain embodiments, and technetium^{99m} and indium¹¹¹ are also often preferred due to their low energy and suitability for long-range detection.

Radioactively labeled proteins or peptides may be produced according to well-known methods in the art. Intermediary functional groups which are often used to bind radioisotopes which exist as metallic ions to peptides include diethylenetriaminepentaacetic acid (DTPA), DOTA, NOTA, porphyrin chelators and ethylene diaminetetracetic acid (EDTA). Also contemplated for use are fluorescent labels, including rhodamine, fluorescein isothiocyanate and renographin.

In certain embodiments, the proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In alternative embodiments, macromolecules of a Be complex may be tagged with a fluorescent marker.

In various embodiments, labels of use may comprise alternative metal nanoparticles other than Be. Methods of preparing nanoparticles are known. (See e.g., U.S. Pat. Nos. 6,054,495; 6,127,120; 6,149,868; Lee and Meisel, J. Phys. Chem. 86:3391-3395, 1982.) Nanoparticles may also be obtained from commercial sources (e.g., Nanoprobes Inc., Yaphank, N.Y.; Polysciences, Inc., Warrington, Pa.). Modified nanoparticles are available commercially, such as Nanogold® nanoparticles from Nanoprobes, Inc. (Yaphank, N.Y.). Functionalized nanoparticles of use for conjugation to proteins or peptides may be commercially obtained.

Pharmaceutical Compositions

In some embodiments, a Be complex and/or one or more other therapeutic agents may be administered to a subject, such as a subject with cancer. Such agents may be administered in the form of pharmaceutical compositions. Generally, this will entail preparing compositions that are essentially free of impurities that could be harmful to humans or animals. One skilled in the art would know that a pharmaceutical composition can be administered to a subject by various routes including, for example, orally or parenterally, such as intravenously.

In certain embodiments, an effective amount of a therapeutic agent must be administered to the subject. An “effective amount” is the amount of the agent that produces a desired effect. An effective amount will depend, for example, on the efficacy of the agent and on the intended effect. An effective amount of a particular agent for a specific purpose can be determined using methods well known to those in the art.

Chemotherapeutic Agents

In certain embodiments, chemotherapeutic agents may be administered. Anti-cancer chemotherapeutic agents of use include, but are not limited to, 5-fluorouracil, bleomycin, busulfan, camptothecins, carboplatin, chlorambucil, cisplatin (CDDP), cyclophosphamide, dactinomycin, daunorubicin, doxorubicin, estrogen receptor binding agents or combination thereof.

Chemotherapeutic agents and methods of administration, dosages, etc., are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts). Some variation in dosage will necessarily occur depending on the condition of the subject being treated. The person responsible for administration will, in any event, determine the appropriate dose for the individual subject.

Hormones

Corticosteroid hormones can be used in a Be complex or in addition to the administration of a Be complex to a subject. Hormones increase the effectiveness of other chemotherapy agents, and consequently, they are frequently used in combination treatments. Prednisone and dexamethasone are examples of corticosteroid hormones. Progestins, such as hydroxyprogesterone caproate, medroxyprogesterone acetate, and megestrol acetate, have been used in cancers of the endometrium and breast. Estrogens such as diethylstilbestrol and ethinyl estradiol have been used in cancers such as prostate cancer. Antiestrogens such as tamoxifen have been used in cancers such as breast cancer. Androgens such as testosterone propionate and fluoxymesterone have also been used in treating breast cancer.

In certain embodiments, anti-angiogenic agents, and/or immunomodulating agents may be used as a component of a Be complex for example angiostatin, anti-VEGF antibodies, anti-PlGF peptides and antibodies, anti-vascular growth factor antibodies, fibronectin peptides, plasminogen activator inhibitors, tissue metalloproteinase inhibitors, interferons, interleukin-12, IP-10, Gro-β, thrombospondin, 2-methoxyoestradiol; cytokines, stem cell growth factors, lymphotoxins; and hematopoietic factors, such as interleukins, colony-stimulating factors, interferons (e.g., interferons-α, -β and -γ), IL-2, IL-6, IL-10, IL-12, IL-18, IL-21, interferon-gamma, TNF-alpha, lipid mediators (eg. leukotrienes or prostaglandins) or combination thereof.

Kits

Various embodiments may concern kits containing components suitable for treating or diagnosing disease or detecting infiltration by an agent in tissue of a patient. Exemplary kits may contain at least one Be complexed to a macromolecule. If the composition containing components for administration is not formulated for delivery via the alimentary canal, such as by oral delivery, a device capable of delivering the kit components through some other route may be included. One type of device, for applications such as parenteral delivery, is a syringe that is used to inject the composition into the body of a subject. Inhalation devices may also be used.

The kit components may be packaged together or separated into two or more separate containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

In one particular embodiment, a kit may include components for a BeLPT assay and a container of Be complexed to a macromolecule. In a more particular embodiment, a kit may include components for a BeLPT assay and a container of Be-ferritin such as ¹⁰Be or ⁷Be.

The embodiments are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### EXAMPLES

### Example 1

In one exemplary method, the affects of a Be complex of Be-ferritin was tested on BAL macrophages (Sawyer et. al. Beryllium-Ferritin: Lymphocyte Prolifieration and Macrophage Apoptosis in Chronic Bervllium Disease, Am J. Respir. Cell. Mol. Biol. vol 31, page 479-477 2004 and the online supplement entitled Beryllium-Ferritin: Lymphocyte Prolifieration and Macrophage Apoptosis in Chronic Beryllium Disease are incorporated herein in their entirety). In this method an increased amount of BAL macrophages from CBD subjects with apoptotic fragmented nuclei after exposure to 100 μM BeSO₄ (50%±6%, mean±SEM, p<0.05 Wilcoxon rank sum test versus the unstimulated control level of nuclear fragmentation <3%), and to a Be-ferritin adduct (40%±2%, p<0.05 versus the ferritin alone treated control<3%) was observed. Based on the binding of carrier-free ⁷BeCl₂ to ferritin, the calculations illustrate that the 50 μg of Be-ferritin adduct used to induce lung macrophage apoptosis contained 270 picomoles of Be. Sub-cellular distribution studies showed that ⁷Be-ferritin localized principally to the cytoplasm of CBD BAL macrophages. Be-ferritin induced apoptosis occurred in BAL macrophages from subjects with BeS (25%±3%) and, in the H36.12J hybrid macrophage cell line (15%±2%). Of interest, BeSO₄ and Be-ferritin did not induce the apoptosis of BAL lymphocytes from CBD and BeS subjects. Macrophages can take up Be-ferritin and can process the Be into Be-antigen in association with MHC class II surface molecules. This complex ligates Be-specific T cell receptors on Be-specific CBD BAL T cells triggering their proliferation. One observation was that Be-ferritin stimulated Be-specific CBD BAL T cell proliferation at concentrations that were 5-6 logs lower than the amounts of BeSO₄ needed to induce comparable results.

Thus, lung cells such as macrophages are capable of taking up Be-ferritin and delivering physiologically relevant levels of Be complex. These complexes are capable of promoting Be-antigen presentation and macrophage apoptosis. Additionally, Be-ferritin is capable of introducing Be to the macrophage's exogenous antigen processing pathway triggering proliferation of Be-specific CBD BAL T cells. Be-specific lung T cells have been demonstrated to fail to undergo clonal deletion after Be-ferrifin exposure. This can result in persistent Be-antigen, Be-specific T cell clonal expansion and cytokine production, and this potentially explains the chronicity of CBD and its ability to develop even after environmental Be exposure has ceased.

This study illustrates the usefulness of a Be-ferritin adduct in defining the molecular mechanisms of Be-induced apoptosis in macrophages. It also describes a Be-ferritin adduct as a potent and physiologically relevant chemical form of the environmental toxin Be. This example illustrates that very low, such as picomolar levels of the environmental toxin Be, when complexed with a host protein such as ferritin, is able to induce macrophage apoptosis and Be-specific CBD BAL T cell proliferation, at concentrations 5-6 logs lower than the amounts of BeSO., needed to elicit similar results. Be-ferritin is capable of being detected in sub-cellular macrophage compartments such as the cytoplasm and nucleus, using gamma counting methods. Although Be sub-cellular compartments such as the cytoplasm and nucleus, sub-cellular components such as microsomes, lysosomes or other internal or external.

These observations indicated 10BAPS (¹⁰Beryllium-Adduct Probes) and other Be complexes can be used to trace intracellular pathways at very low levels of radioisotope. These methods can be used to study sub-cellular components. For example, such intracellular components can include studying mitochondrial or lysosomal associated biomolecules and processes.

### Example 2

In another exemplary method, 10BAPs (¹⁰Be-Adduct Probes) were designed to trace intracellular pathways using low levels of the radioisotope ¹⁰Be. The ¹⁰Be-Adduct Probes 10BAPs are unique ¹⁰Be-Adduct formed with proteins, lipids, carbohydrates, polysaccharides, nucleic acids, complex and simple organic and inorganic compounds and molecules—that when coupled to a detection system such as AMS analysis can be used to follow the interaction of these ¹⁰Be-Adduct Probes_with host tissues, cells, sub-cellular and molecular components.

¹⁰Be-Adduct Probes 10BAPs can be detected in association with these host components at levels as low as 1×10⁻¹⁸ M by AMS and at levels of the 10BAPs that are not achievable using current, conventional physical-chemical analysis.

Current techniques indicate elemental, non-radioactive, stable, ⁹Be can be detected using atomic absorption spectroscopy and to some extent mass spectroscopy. However, these methods of detection can require that a high level of ⁹Be is present, 10 μM, and that the sample size be might be greater than a milligram of sample, to provide a sufficient amount of ⁹Be for detection. Radioactive ⁷Be (gamma particle=0.48 Mev) can be detected by gamma-counting, levels of detection can be limited to Be concentrations greater than 10-100 μM ⁷Be.

In one exemplary detection technique, Accelerator Mass Spectroscopy, AMS, can measure the ratio of ¹⁰Be/⁹Be at ¹⁰Be levels of 1×10⁻¹⁸ M. AMS has been adapted to overcome the limitations of detection sensitivity of long-lived radionuclides that typically can not be analyzed with decay counting or conventional mass spectrometry. AMS is extremely sensitive for counting atomic nuclei, is able to detect approximately 1×10⁻¹⁵ to 1×10⁻¹⁸ M Be, in smaller sample sizes, for example a hundred or even thousand-fold smaller as compared to decay counting.

In another exemplary method, a Be complex can be designed to include a macromolecule that detects a toxic molecule, a virus, a predetermined compound such as a bacterial produced toxin, a flu or SARS virus or a pre-determined compound associated with a disease or condition in a subject or a sample. Thus, using a sensitive system such as AMS, it becomes possible to now detect atoms of Be associated with the sample or subject in low sample amounts and at very low levels of 10BAP exposure not previously achievable.

Materials and Methods

Chemicals and Reagents: Carrier-free ⁷BeCl₂, specific activity 2.6 mCi/mg at 1.66 mCi/ml, was purchased from Oak Ridge National Laboratory (Oak Ridge, Tenn.) and counts per minute determined using a Packard Cobra Auto-gamma counter (Downers Grove, Ill.). Ferritin was purchased from Sigma Chemical Co. (St. Louis, Mo.). Beryllium sulfate (Brush Wellman, Inc., Cleveland, Ohio) and aluminum sulfate (Sigma) were maintained at 4° C. as stock solutions of 1×10⁻³ M BeSO₄ or 1×10⁻³ M Al₂(SO₄)₃ in water and diluted 1:10 or 1:100 during cell culture for final concentrations of 100 μM an 10 μM respectively. Phytohemagglutinin (PHA, Sigma) was used as a positive control for T cell proliferation in the clinical Be lymphocyte proliferation test (BeLPT) [14]. Phycoerythrin-labeled anti-CD95, FITC anti-CD71, FITC anti-CD4 and their corresponding labeled isotype control antibodies were purchased from BD-Biosciences (San Diego, Calif.).

Beryllium-ferritin adducts were prepared as described previously. One mg of ferritin in 1 ml of 0.2 M tris-acetate, pH 6.5, plus 0.1 M BeSO₄ was incubated for 15 min at 37° C. One mg of unlabeled ferritin served as the protein control and an equal volume of 0.1 M BeSO₄ that was dialyzed in the absence of protein served as the “dialysis control.” Ferritin, Be-ferritin and 0.1 M BeSO₄ were dialyzed in 2×1 L of 0.02 M tris-acetate, pH=6.5, for 12 hr each, at 4° C., then transferred to sterile tubes and held at 4° C. until use.

Cell Cultivation: Bronchoalveolar lavage (BAL) was performed as previously described. Cells retrieved from the lung by BAL were cultured in complete medium (RPMI 1640 medium (Cambrex Bioproducts, Walkersville, Md.) containing 10% iron supplemented calf serum (Hyclone, Logan, Utah). 0.29 mg/ml L-glutamine, 100 U/ml penicillin G and 100 μg/ml streptomycin sulfate). BAL macrophages and T cells were separated by an adherence method known in the art.

H36.12j cells (ATCC, CRL 2449) are clonally derived hybrid precursor macrophages derived from the fusion of drug selected P388D.1 (DBA/2, H2^{d}) macrophages with percoll gradient purified, proteose peptone elicited macrophages obtained from C57B1/6N (H2^{b}) mice. H36.12j cells were cultivated in Dulbecco's Modified Eagle's medium (BioWhittiker, Walkersville, Md.) supplemented with 10% heat inactivated calf serum, 0.29 mg/ml L-glutamine, 100 U/ml penicillin G, and 100 μg/ml streptomycin sulfate.

BeLPT: For clinical evaluation of Be sensitization, as presented in Table 1, the blood and BAL beryllium lymphocyte proliferation tests (BeLPT) were performed according to a clinical assay described by Mroz et al. Blood and BAL cells were adjusted to a concentration of 1×10⁶ per ml of complete medium and 200 μl aliquots per well were then cultured in triplicate samples per treatment. Three plates were prepared in which the cells were unstimulated or exposed to 100 μM and 10 μM BeSO₄, 100 μM Al₂(SO₄)₃, PHA, 50 μl of “dialysis contro,” 50 μg of ferritin and 50 μg of the Be-ferritin adduct. The plates were incubated, as above, and on days 4, 5 and 6 the cultures were pulse labeled with the DNA specific precursor tritiated thymidine deoxyriboside (³HTdR; [methyl-³H]-Thymidine, specific activity=5.0 Ci/mmole; Amersham Biosciences, Piscataway, N.J.) for 4 hr at 37° C. in an atmosphere containing 5% CO₂. ³H-DNA was harvested onto glass fiber filters using a Tomtec 96 well plate harvester, and the glass fiber filters were counted in a Packard TopCount NXT liquid scintillation counter (Packard Inst. Co., Meriden, Conn.). Thymidine uptake for the unstimulated controls on days 4, 5 and 6 are normally in the range of 150 cpm to 500 cpm. For the clinical evaluation of blood and BAL T cell proliferation in response to BeSO₄ stimulation shown in Table 1, the mean (±SEM) peak stimulation index (SI) for thymidine uptake was reported as the ratio of the test sample counts per minute (cpm) to the cpm in the unstimulated (medium alone) control.

Statistics: Repeated Measures ANOVA was used to determine the effect of treatments while adjusting for the variability of subjects. In cases where there was also a time variable, a doubly repeated measures model was used. After the data were checked for significant treatment differences, individual contrasts were calculated to compare treatment means of interest. Normalizing transformations were made in cases where the data were non-Gaussian. When data transformations were unsuccessful, suitable nonparametric tests were substituted for parametric tests.

Study Population: The clinical characteristics of the BeS and CBD study subjects (Table 1) reflected a beryllium work force. None of the BeS subjects, and 6/8 CBD subjects were currently using oral glucocorticosteroids. Six of the eight CBD subjects were former smokers, one a never smoker, and one individual was a current smoker. Thirteen of the nineteen BeS subjects were former smokers and 6/19 never smokers.

CBD subjects had significantly higher total number of BAL WBCs (43×10⁶: range; 16-118, p<0.05) in comparison to BeS subjects (25×10⁶: range 8-50) reflecting a significant increase in the absolute number of CBD BAL lymphocytes, 25%±12% (mean±SEM, p<0.05).

The CBD and BeS subjects enrolled in this study met the clinical case definitions based on the proliferation of blood and BAL T cells in the clinical BeLPT. Both BeS and CBD subjects' blood BeLPT stimulation index (SI) were significantly increased at a median 3.4 (range 1.1-30) and median 9.2 (range 1.1-17) respectively. Only the CBD subjects had significantly increased BAL BeLPT SI=median 95 (range 1.2-190, p<0.05) reflecting an increase in the numbers of Be-specific CD4⁺ effector-memory T cells present in the CBD BAL mixed cell compartment.

Radio-labeled ⁷Be-ferritin and calculated translocation: Based on standard calculations using a ⁷Be T_{1/2} of 53 days, the amount of ⁷Be associated with 50 μg of the ⁷Be-ferritin adduct detected was 270 picomoles (2.7×10⁻¹⁰ M=Be-ferritin): 1) The cpm/count efficiency (60 min)(24 hr)=dpm day ⁻¹/λ=Be atoms/molecule of ferritin. 2) The atoms of Be/molecule of ferritin (molecules of ferritin in 50 μg of protein)/Avogadro's number/volume=moles of beryllium.

### Example 3

Materials and Methods for Table 1

Study Population: Nineteen CBD and eight beryllium sensitized (BeS) patients were consecutively enrolled in this study based on the availability of BAL samples. The diagnosis of CBD had been previously established using defined criteria including of a history of Be-exposure, the presence of granulomatous inflammation on lung biopsy and a positive proliferation response of blood and/or BAL T cells to Be-stimulation in vitro. The BeS patients had a history of Be-exposure, normal lung histology on lung biopsy and a positive proliferation response of blood T cells to Be-stimulation in vitro.

Chemicals and Reagents: Carrier-free ⁷BeCl₂, specific activity 2.6 mCi/mg at 1.66 mCi/ml, was purchased from Oak Ridge National Laboratory (Oak Ridge, Tenn.) and counts per minute determined using a Packard Cobra Auto-gamma counter (Downers Grove, Ill.). Ferritin, transferrin, lactoferrin, ceruloplasmin and metallothionein were purchased fiom Sigma Chemical Co. (St. Louis, Mo.). Beryllium sulfate (Brush Wellman, Inc., Cleveland, Ohio) and aluminum sulfate (Sigma) were maintained at 4° C. as stock solutions of 1 mM BeSO₄ or 1 mM Al₂(SO₄)₃ in water and dilute 1:IOor 1:100 during cell culture for final concentrations of 100 μM an 100 μM respectively.

Be-ferritin adducts were prepared as previously described.

Cell Cultivation: Bronchoalveolar lavage (BAL) was performed as previously described. BAL cells were cultured as described, in complete medium (RPMI 1640 medium (BioWhittaker, Walkersville, Md.) containing 10% iron supplemented calf serum (Hyclone, Logan, Utah). 0.29 mg/ml L-glutamine, 100 U/ml penicillin and 100 mg/ml streptomycin). For all studies cells were enumerated by hemocytometer and viability was greater than 90% at the initiation of each experiment.

Beryllium lymphocyte proliferation test (BeLPT): The BeLPT was performed by the method of Mroz et. al. [4]. Statistics: Analysis of variance was performed using IMP software. Positive values for pairs of means were considered significantly different at a p<0.05 using.

Study Population: The clinical characteristics of the BeS and CBD study subjects (Table 2) reflected a beryllium work-force as described previously. None of the BeS subjects and 6/8 CBD subjects were currently using steroids. CBD subjects had significant increased total BAL WBCs (43×10⁶: minimum 16; maximum 118, p<0.05) in comparison to BeS subjects (25×50) reflecting a significant increase in the absolute number of CBD BAL lymphocytes, 25+12% (mean±SEM, p<0.05). Both BeS and CBD subject's blood BeLPT stimulation index (SI) were significantly increased at a median 3.4 (minimum 1.1; maximum 30) and median 9.2 (minimum 1.1; maximum 17) respectively (Table 1). Only CBD subjects had significantly increased BAL BeLPT SI=median 95 (mininum 1.2; maximum 190).

TABLE 2
Clinical characteristics of the BeS (n = 8) and
CBD (n = 19) study subjects.

| | BeS | CBD |
|---|---|---|
| Age (media yr; minimum, maximum) | 56 (37, 76) | 50 (30, 61) |
| Gender (F/M) | 3/16 | 1/7 |
| Current Steroid Use (yes/no) | 0/19 | 6/8 |
| Total BALWBCX10⁶ | | |
| (median; minimum, maximum) | 25 (8, 50) | 43 (16, 118)* |
| % Macrophages (mean ± SEM) | 83 ± 4 | 74 ± 11 |
| % Lymphocytes (mean + SEM) | 15 ± 3 | 25 + 12* |
| % Neutrophils (mean ± SEM) | 1.2 + 0.4 | 0.1 + 0.06 |
| Blood BeLPT SI (median; min, max) | 3.4 (1.1, 30)* | 9.2 (1.1, 17)* |
| BAL BeLPT SI (median; min, max) | 1.9 (1.2, 46) | 95 (1.2, 190)* |

*p < 0.05, Tukey Kramer

TABLE 3
Peak CBD BAL cell BeLPT stimulation index (n = 4)

| | STIMULATION INDEX |
|---|---|
| TREATMENT | median (minimum, maximum) |
| 10⁻¹⁰M Be-FERRITIN | 3.8 (2.8, 20.3)* |
| 10⁻⁴M BeSO, | 18.5 (3.7, 124)** |
| 10⁻⁴M Al₂(SO₄)₃ | 0.4 (0.24, 0.4) |
| FERRITIN | 1.1 (0.8, 1.3) |
| DIALYSIS CONTROL | 1.3 (1.1, 1.5) |

*p < 0.05 versus ferritin, aluminum sulfate and the dialysis control
**p < 0.05 versus aluminum sulfate and the dialysis control

All of the COMPOSITIONS and/or METHODS and/or APPARATUS disclosed and claimed herein can be made and executed without undue experimentation in light of the present disclosure. While the compositions and methods of this invention have been described in terms of preferred embodiments, it will be apparent to those of skill in the art that variation may be applied to the COMPOSITIONS and/or METHODS and/or APPARATUS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of use comprising:
a) obtaining a Be complex;
b) administering the Be complex to a sample or a subject; and
c) detecting the Be complex and/or a byproduct of the Be complex in the sample or subject.

2. The method of claim 1, wherein administering the Be complex to the sample comprises administering the Be complex to one or more cells, tissues, organs or combination thereof.

3. The method of claim 1, further comprising identifying a receptor that binds to the Be complex.

4. The method of claim 1, further comprising identifying a sub-cellular fraction, cell, tissue or organ to which the Be complex localizes.

5. The method of claim 4, further comprising measuring the level of Be complex in the sub-cellular fraction, cell, tissue or organ.

6. The method of claim 5, further comprising measuring the rate of clearance of the Be complex from the sub-cellular fraction, cell, tissue or organ.

7. The method of claim 6, wherein obtaining a Be complex comprises obtaining a Be complex selected from the group consisting of obtaining a ⁹Be complex, obtaining a ¹⁰Be complex, obtaining a ⁷Be complex or combination thereof.

8. A method of use comprising:
a) obtaining a ¹⁰Be complex;
b) administering the Be complex to a sample or a subject; and
c) measuring the ratio of ¹⁰Be to ⁹Be.

9. The method of claim 8, wherein the ratio of ¹⁰Be/⁹Be is measured by accelerator mass spectroscopy (AMS).

10. The method of claim 8, wherein administering the ¹⁰Be complex to the sample comprises administering the ¹⁰Be complex to one or more cells, tissues, organs or combination thereof.

11. The method of claim 8, further comprising identifying a receptor that binds to the ¹⁰Be complex.

12. The method of claim 8, further comprising identifying a sub-cellular fraction, cell, tissue or organ to which the ¹⁰Be complex localizes.

13. The method of claim 12, further comprising measuring the level of ¹⁰Be complex in the sub-cellular fraction, cell, tissue or organ.

14. The method of claim 13, further comprising measuring the rate of clearance of the ¹⁰Be complex from the sub-cellular fraction, cell, tissue or organ.

15. A method of use comprising:
a) obtaining a beryllium complex; and
b) inducing T-cell proliferation with the beryllium complex in a beryllium lymphocyte proliferation test (BeLPT).

16. The method of claim 15, wherein the beryllium is ¹⁰Be or ⁷Be.

17. The method of claim 15, wherein the beryllium is non-radioactive beryllium.

18. The method of claim 15, wherein the complex comprises beryllium and a macromolecule.

19. The method of claim 15, wherein the macromolecule is ferritin, lactoferrin, transferrin, metallothionein or ceruloplasmin.

20. The method of claim 15, wherein the BeLPT test is performed at a Be concentration that is at least an order of magnitude lower than the concentrations of inorganic salts of beryllium used to perform BeLPT assays.

21. The method of claim 15, wherein the BeLPT test is performed at a Be concentration that is at least three orders of magnitude lower than the concentrations of inorganic salts of beryllium used to perform BeLPT assays.

22. The method of claim 15, wherein the concentration of the Be-complex in the BeLPT assay is less than 1 micromolar.

23. The method of claim 15, wherein the concentration of the Be-complex in the BeLPT assay is less than 1 nanomolar.

24. A method of use comprising:
a) obtaining a complex comprising ¹⁰Be or ⁷Be, a macromolecule and one or more pharmaceutical agent(s) that is attached to the complex; and
b) providing the complex to a subject in need of the pharmaceutical agent.

25. The method of claim 24, further comprising attaching the macromolecule directly to ¹⁰Be or ⁷Be.

26. The method of claim 24, further comprising:
i) delivering the complex to a target cell that has a receptor for the macromolecule; and
ii) allowing the complex to bind to the receptor.

27. The method of claim 26, further comprising internalizing the one or more pharmaceutical agent(s) by receptor mediated uptake of the complex.

28. The method of claim 24, wherein the macromolecule is selected from the group consisting of a protein, peptide, virus, prion, nucleic acid, lipid, polysaccharide, pharmaceutical agent, carbohydrate, organic compound and inorganic compound.

29. A beryllium complex composition comprising:
a) ¹⁰beryllium (¹⁰Be) or ⁷beryllium (⁷Be)
b) a macromolecule attached to the beryllium; and
c) one or more therapeutic agent(s) attached to the beryllium and/or to the macromolecule.

30. The composition of claim 29, wherein the macromolecule is a protein, peptide, virus, prion, nucleic acid, lipid, polysaccharide, pharmaceutical agent, carbohydrate, organic compound or inorganic compound.

31. The composition of claim 29, wherein the macromolecule is ferritin, lactoferrin, transferrin, metallothionein or ceruloplasmin.

32. The composition of claim 29, wherein the macromolecule is an antibody or an antibody fragment.

33. The composition of claim 29, further comprising

34. A kit comprising:
a) a composition comprising a beryllium complex
b) a delivery device.
